(19) United States
(12) Patent Application Publication
(10) Pub. No.: US 2021/0295214 A1
(43) Pub. Date: Sep. 23, 2021

(54) LEARNING APPARATUS, LEARNING METHOD AND COMPUTER PROGRAM

(71) Applicant: NIPPON TELEGRAPH AND TELEPHONE CORPORATION, Tokyo (JP)

(72) Inventor: Yohei KATAYAMA, Musashino-shi, Tokyo (JP)

(73) Assignee: NIPPON TELEGRAPH AND TELEPHONE CORPORATION, Tokyo (JP)

(21) Appl. No.: 17/261,140

(22) PCT Filed: Jun. 28, 2019
(86) PCT No.: PCT/JP2019/025846

§ 371 Date: Jan. 18, 2021

(30) Foreign Application Priority Data
Jul. 26, 2018 (JP) 2018-140113

(51) Int. Cl. G06N 20/00; G06N 5/04; A61B 5/01; A61B 5/16; A61B 5/00; A61B 5/369; A61B 5/022

(52) U.S. Cl. CPC G06N 20/00; G06N 5/048; A61B 5/01; A61B 5/165; A61B 2560/0242; A61B 5/163; A61B 5/369; A61B 5/022; A61B 5/7264

(57) ABSTRACT

A learning apparatus including a biological information acquisition unit that acquires biological information, the biological information being information indicating a vital reaction of a test subject to a predetermined environment, an emotional information acquisition unit that acquires emotional information, the emotional information being information indicating an emotion of the test subject toward the environment, a first environmental information acquisition unit that acquires environmental information, the environmental information being information indicating an attribute of the environment acting on the test subject, and a relationship information learning unit that learns, through machine learning, a relationship among the biological information, the emotional information, and the environmental information based on the biological information, the emotional information, and the environmental information.

### TECHNICAL FIELD

The present invention relates to a learning apparatus, a learning method, and a computer program.

### BACKGROUND ART

As a framework for forming (learning) a policy (hereinafter referred to as a “control selection policy”) of selecting a highly evaluated process by repeatedly evaluating a result of a process previously selected by a system itself, reinforcement learning has been devised (see Non-Patent Literature 1). A system that executes reinforcement learning will be referred to as a reinforcement learning system below. In order to enhance the accuracy of a control section policy in reinforcement learning, the number of times of learning by a reinforcement learning system needs to be increased. Note that the accuracy of a control selection policy means the probability that a highly evaluated process is selected in a reinforcement learning system. Namely, this means that the higher the probability that a highly evaluated process is selected is and the higher evaluation results of such processes receive, the higher the accuracy is.

### CITATION LIST

### Non-Patent Literature

Non-Patent Literature 1: Takaki Makino et al., “Korekara no kyoka gakushu” (reinforcement learning in future), 1st imp. of 1st ed., Morikita Publishing Co., Ltd., Oct. 31, 2016

### SUMMARY OF THE INVENTION

### Technical Problem

In general, a value called a reward is present in reinforcement learning. A reward is a value indicating how a result of a process previously executed by a reinforcement learning system is evaluated. In a case where an evaluation criterion is clear as in determination of the win or loss of a game, it is easy for the reinforcement learning system to determine a reward value. In contrast, in a case where an evaluation criterion close to a human sensibility is needed as in determination as to whether a luxury grocery item is good or bad, it is not easy for the reinforcement learning system to determine a reward value. For this reason, in a conventional reinforcement learning system, a designer of the reinforcement learning system observes a relationship between a reward and the accuracy of a control selection policy and evaluates a learning result based on the sensibility of the designer itself, thereby forming a high-accuracy control selection policy. More specifically, in the conventional reinforcement learning system, a high-accuracy control section policy has been formed by the designer's updating, through learning, a combination of a reward function that determines a reward based on a result of a process selected by an accuracy control selection policy and the control selection policy (see FIG. 8). For this reason, the designer needs to observe a relationship between a reward and the accuracy of a control selection policy on each learning occasion until a desired control selection policy is formed, and labor of the designer may increase with increase in the accuracy of a control selection policy.

Under the above-described circumstances, an object of the present invention is to provide a learning apparatus, a learning method, and a computer program capable of curbing increase in labor of a designer required to form a control selection policy in reinforcement learning that needs an evaluation criterion close to a human sensibility.

### Means for Solving the Problem

According to one aspect of the present invention, there is provided a learning apparatus including a biological information acquisition unit that acquires biological information, the biological information being information indicating a vital reaction of a test subject to a predetermined environment, an emotional information acquisition unit that acquires emotional information, the emotional information being information indicating an emotion of the test subject toward the environment, a first environmental information acquisition unit that acquires environmental information, the environmental information being information indicating an attribute of the environment acting on the test subject, and a relationship information learning unit that learns, through machine learning, a relationship among the biological information, the emotional information, and the environmental information based on the biological information, the emotional information, and the environmental information.

According to one aspect of the present invention, there is provided a learning apparatus including an output unit that acts on a predetermined environment, a control unit that controls operation of the output unit, a second environmental information acquisition unit that acquires environmental information, the environmental information being information indicating an attribute of the environment, and a reward output unit that outputs, based on the environmental information indicating the attribute of the environment acted on by the output unit and relationship information stored in advance in the learning apparatus and indicating a relationship among biological information that is information indicating a vital reaction of a test subject to the environment, environmental information that is information having a one-to-one relationship with the biological information and indicating the attribute of the predetermined environment acting on the test subject, and emotional information that is information having a one-to-one relationship with the biological information and indicating an emotion of the test subject toward the environment, a numerical value that is represented based on the emotional information and indicates magnitude of the emotion of the test subject, wherein the control unit updates a value of a control parameter for controlling the operation of the output unit based on the numerical value.

According to one aspect of the present invention, there is provided a learning apparatus including a biological information acquisition unit that acquires biological information, the biological information being information indicating a vital reaction of a test subject to a predetermined environment, an emotional information acquisition unit that acquires emotional information, the emotional information being information indicating an emotion of the test subject toward the environment, a first environmental information acquisition unit that acquires environmental information, the environmental information being information indicating an attribute of the predetermined environment acting on the test subject, a relationship information learning unit that learns, through machine learning, a relationship among the biological information, the emotional information, and the environmental information based on the biological information, the emotional information, and the environmental information, a control unit that controls operation of the output unit, and a reward output unit that outputs a numerical value that is represented based on the emotional information and indicates magnitude of the emotion of the test subject, based on the environmental information indicating the attribute of the environment acted on by the output unit and relationship information that is information stored in advance in the learning apparatus and indicating a one-to-one relationship among the biological information, the environmental information, and the emotional information, wherein the control unit updates a value of a control parameter for controlling the operation of the output unit based on the numerical value.

According to one aspect of the present invention, in the above-described learning apparatus, the relationship information learning unit further learns a relationship between the biological information that has a predetermined degree or a higher degree of correlation with the emotional information and the emotional information.

According to one aspect of the present invention, there is provided a learning method including a biological information acquisition step of acquiring biological information that is information indicating a vital reaction of a test subject to a predetermined environment, an emotional information acquisition step of acquiring emotional information that is information indicating an emotion of the test subject toward the environment, a first environmental information acquisition step of acquiring environmental information that is information indicating an attribute of the environment acting on the test subject, and a relationship information learning step of learning, through machine learning, a relationship among the biological information, the emotional information, and the environmental information based on the biological information, the emotional information, and the environmental information.

According to one aspect of the present invention, there is provided a learning method including a control step of controlling operation of an output unit that acts on a predetermined environment, a second environmental information acquisition step of acquiring environmental information that is information indicating an attribute of the environment, and a reward output step of outputting, based on the environmental information indicating the attribute of the environment acted on by the output unit and relationship information stored in advance in a learning apparatus and indicating a relationship among biological information that is information indicating a vital reaction of a test subject to the environment, environmental information that is information having a one-to-one relationship with the biological information and indicating the attribute of the predetermined environment acting on the test subject, and emotional information that is information having a one-to-one relationship with the biological information and indicating an emotion of the test subject toward the environment, a numerical value that is represented based on the emotional information and indicates magnitude of the emotion of the test subject, wherein, in the control step, a value of a control parameter for controlling the operation of the output unit is updated based on the numerical value.

According to one aspect of the present invention, there is provided a learning method including a biological information acquisition step of acquiring biological information that is information indicating a vital reaction of a test subject to a predetermined environment, an emotional information acquisition step of acquiring emotional information that is information indicating an emotion of the test subject toward the environment, a first environmental information acquisition step of acquiring environmental information that is information indicating an attribute of the predetermined environment acting on the test subject, a relationship information learning step of learning, through machine learning, a relationship among the biological information, the emotional information, and the environmental information based on the biological information, the emotional information, and the environmental information, a control step of controlling operation of an output unit that acts on the environment, and a reward output step of outputting a numerical value that is represented based on the emotional information and indicates magnitude of the emotion of the test subject, based on the environmental information indicating the attribute of the environment acted on by the output unit and relationship information that is information stored in advance in a learning apparatus and indicating a one-to-one relationship among the biological information, the environmental information, and the emotional information, wherein, in the control step, a value of a control parameter for controlling the operation of the output unit is updated based on the numerical value.

According to one aspect of the present invention, there is provided a computer program for causing a computer to function as the above-described learning apparatus.

### Effects of the Invention

According to the present invention, it is possible to curb increase in labor of a designer required to form a control selection policy if an evaluation criterion close to a human sensibility is needed.

### BRIEF DESCRIPTION OF DRAWINGS

FIG. 1 is a diagram showing a specific example of a system configuration of a learning system 1 according to a first embodiment.

FIG. 2 is a flowchart showing the flow of a specific process by a first learning apparatus 10 according to the first embodiment.

FIG. 3 is a flowchart showing the flow of a specific process by a second learning apparatus 20 according to the first embodiment.

FIG. 4 is a diagram showing an example of application in which the learning system 1 according to the first embodiment is applied to learning of cooking by a cooking robot.

FIG. 5 is a diagram showing a specific example of a system configuration of a learning system 1a according to a second embodiment.

FIG. 6 is a flowchart showing the flow of a specific process by a third learning apparatus 30 according to the second embodiment.

FIG. 7 is a diagram showing an example of application in which the learning system 1a according to the second embodiment is applied to learning of display screen control by an image display device.

FIG. 8 is a diagram showing a specific example of a learning system as a conventional example.

### DESCRIPTION OF EMBODIMENTS

### First Embodiment

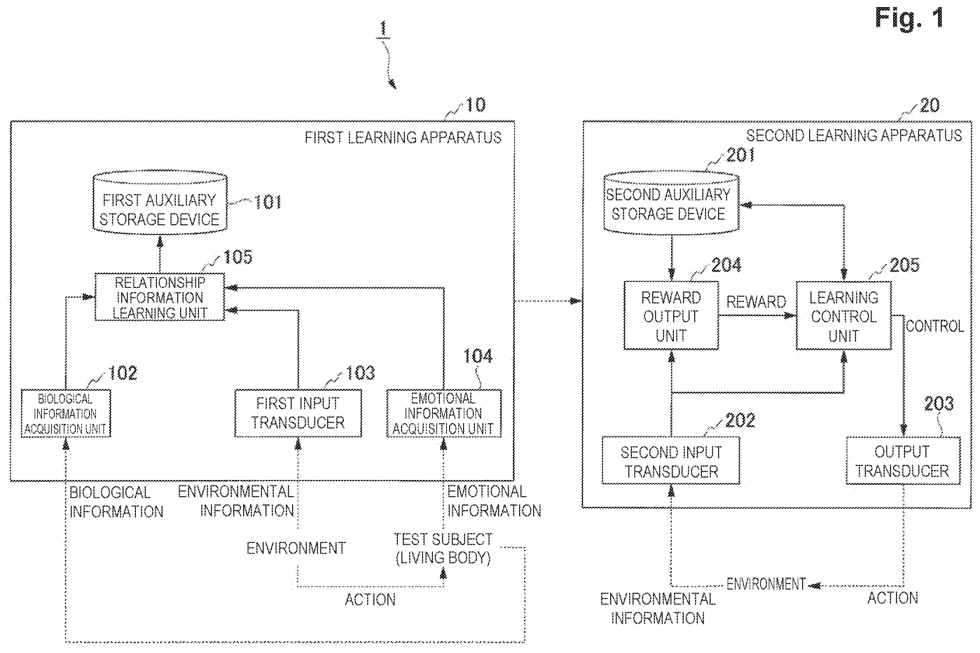

FIG. 1 is a diagram showing a specific example of a system configuration of a learning system 1 according to a first embodiment.

The learning system 1 includes a first learning apparatus 10 and a second learning apparatus 20.

The first learning apparatus 10 acquires environmental information, biological information, and emotional information. The environmental information is information indicating an attribute of a predetermined environment that acts on a test subject for the learning system 1. The biological information is information indicating a vital reaction of the test subject to the predetermined environment. The emotional information is information indicating an emotion of the test subject toward the environment.

The first learning apparatus 10 learns, based on the acquired environmental information, biological information, and emotional information, a relationship among the environmental information, the biological information, and the emotional information. Note that the environmental information, the biological information, and the emotional information have a one-to-one relationship with one another.

Note that the predetermined environment that acts on the test subject may be any environment. The predetermined environment that acts on the test subject may be, for example, air around the test subject. The predetermined environment that acts on the test subject may be, for example, a dish. The emotional information may indicate any emotion. The emotional information may be, for example, information indicating a like or a dislike.

The first learning apparatus 10 outputs information (hereinafter referred to as “relationship information”) indicating the relationship among the environmental information, the biological information, and the emotional information, which is a learning result, to the second learning apparatus 20. Note that the relationship information is an example of a reward function.

The second learning apparatus 20 acts on the environment. The acting on the environment specifically means that the second learning apparatus 20 produces a change in the environment. The second learning apparatus 20 stores, in advance, the relationship information learned by the first learning apparatus 10. The second learning apparatus 20 stores reinforcement learning data. The reinforcement learning data is a value of a control parameter for controlling the operation of the second learning apparatus 20 of acting on the environment. The reinforcement learning data is a value to be updated at predetermined timing by the second learning apparatus 20.

The second learning apparatus 20 acquires environmental information. The second learning apparatus 20 updates the reinforcement learning data based on the acquired environmental information, the relationship information, and a current value of the reinforcement learning data. The second learning apparatus 20 executes a predetermined operation corresponding to the reinforcement learning data and acts on the environment. Note that the current value means a value immediately before the updating. A predetermined operation that corresponds to the reinforcement learning data and acts on the environment will be referred to as an active operation below.

The first learning apparatus 10 includes a CPU (Central Processing Unit), a RAM (Random Access Memory), a first auxiliary storage device 101, and the like that are connected by a bus and executes a program. The first learning apparatus 10 functions as a device including a biological information acquisition unit 102, a first input transducer 103, an emotional information acquisition unit 104, and a relationship information learning unit 105 through the execution of the program.

The first auxiliary storage device 101 is constructed using a storage device, such as a magnetic hard disk device or a semiconductor storage device. The first auxiliary storage device 101 stores relationship information. If the relationship information is, for example, information representing a relationship among numerical environmental information, numerical biological information, and numerical emotional information and is a predetermined unary expression or polynomial expression, the first auxiliary storage device 101 stores the predetermined unary expression or polynomial expression and a coefficient (coefficients) of the predetermined unary expression or polynomial expression. The numerical environmental information is a value representing contents indicated by environmental information in accordance with a predetermined rule. The numerical biological information is a value representing contents indicated by biological information in accordance with a predetermined rule. The numerical emotional information is a numerical value indicating the magnitude of an emotion of the test subject represented based on emotional information in accordance with a predetermined rule. As for the numerical emotional information, for example, a like is represented by +1, and a dislike is represented by (−1).

The biological information acquisition unit 102 acquires biological information. The biological information acquisition unit 102 may be anything as long as it can acquire predetermined information related to a vital reaction of the test object. The biological information acquisition unit 102 may be a clinical thermometer, for example, if the predetermined vital-reaction-related information is information indicating a change in body temperature. The biological information acquisition unit 102 may be a camera, for example, if the predetermined vital-reaction-related information is information indicating the degree of dilation of a pupil. The biological information acquisition unit 102 may be a taste sensor, for example, if the predetermined vital-reaction-related information is gustatory information. The biological information acquisition unit 102 may be an electroencephalograph, for example, if the predetermined vital-reaction-related information is information indicating brain waves. The biological information acquisition unit 102 may be a sphygmomanometer, for example, if the predetermined vital-reaction-related information is information indicating a change in blood pressure. The biological information acquisition unit 102 may be an ocular movement measurement instrument, for example, if the predetermined vital-reaction-related information is information on ocular movement. The biological information acquisition unit 102 may be a heart rate meter, for example, if vital-reaction-related information is information indicating a heart rate.

The biological information acquisition unit 102 generates a signal indicating the acquired biological information. A signal to be generated by the biological information acquisition unit 102 may be any signal as long as the signal indicates the acquired biological information and may be an electrical signal or an optical signal.

The first input transducer 103 acquires environmental information. The first input transducer 103 may be anything as long as it can acquire predetermined information related to the environment that acts on the test subject. The first input transducer 103 may be a thermometer, for example, if the predetermined environment-related information is information indicating an atmospheric temperature. The first input transducer 103 may be a pressure gauge, for example, if the predetermined environment-related information is information indicating an atmospheric pressure.

The first input transducer 103 may be a hygrometer, for example, if the predetermined environment-related information is information indicating a humidity. The first input transducer 103 may be a salinometer, for example, if the environment is cooking, and the predetermined environment-related information is a salt concentration. The first input transducer 103 may be a saccharimeter, for example, if the environment is cooking, and the predetermined environment-related information is a sugar concentration.

The first input transducer 103 generates a signal indicating the acquired environmental information. A signal to be generated by the first input transducer 103 may be any signal as long as the signal indicates the acquired environmental information and may be an electrical signal or an optical signal.

The emotional information acquisition unit 104 acquires emotional information. The emotional information acquisition unit 104 is configured to include an input device, such as a mouse, a keyboard, or a touch panel. The emotional information acquisition unit 104 may be configured as an interface that connects such input devices to the first learning apparatus 10. The emotional information acquisition unit 104 accepts emotional information input to the first learning apparatus 10.

The relationship information learning unit 105 learns through machine learning relationship information based on biological information, environmental information, and emotional information. The learning of the relationship information through machine learning by the relationship information learning unit 105 specifically means that, if the relationship information is information representing a relationship among numerical environmental information, numerical biological information, and numerical emotional information and is a predetermined unary expression or polynomial expression, the relationship information learning unit 105 determines a coefficient (coefficients) of the unary expression or polynomial expression through machine learning based on the numerical environmental information, the numerical biological information, and the numerical emotional information.

Note that the numerical environmental information may be acquired in any manner based on the environmental information. The numerical environmental information may be acquired by, for example, the first input transducer 103 digitizing contents indicated by the environmental information in accordance with the predetermined rule.

Note that the numerical biological information may be acquired in any manner based on the biological information. The numerical biological information may be acquired by, for example, the biological information acquisition unit 102 digitizing contents indicated by the biological information in accordance with the predetermined rule.

Note that the numerical emotional information may be acquired in any manner based on the emotional information. The numerical emotional information may be acquired by, for example, the emotional information acquisition unit 104 digitizing contents indicated by the emotional information in accordance with the predetermined rule.

The second learning apparatus 20 includes a CPU (Central Processing Unit), a RAM (Random Access Memory), a second auxiliary storage device 201, and the like that are connected by a bus and executes a program. The second learning apparatus 20 functions as a device including a second input transducer 202, an output transducer 203, a reward output unit 204, and a learning control unit 205 through the execution of the program.

The second auxiliary storage device 201 is constructed using a storage device, such as a magnetic hard disk device or a semiconductor storage device. The second auxiliary storage device 201 stores relationship information, a control selection policy, and reinforcement learning data. The control selection policy is a program that causes the second learning apparatus 20 to execute an active operation corresponding to a current value of the reinforcement learning data, using the current value of the reinforcement learning data.

The control section policy may be any program as long as the program causes the second learning apparatus 20 to execute an active operation corresponding to the current value of the reinforcement learning data. The control selection policy may be, for example, a conversion expression that converts the current value of the reinforcement learning data into a control parameter for controlling the output transducer 203 that is described later. In this case, the conversion expression is, for example, a unary expression or polynomial expression that takes the reinforcement learning data as a coefficient (coefficients).

The second input transducer 202 acquires environmental information. The second input transducer 202 may be anything as long as it can acquire environmental information to be acquired by the first input transducer 103. The second input transducer may be anything as long as it can acquire information indicating an atmospheric temperature, for example, if the first input transducer 103 is a thermometer. The second input transducer may be anything as long as it can acquire information indicating an atmospheric pressure, for example, if the first input transducer 103 is a pressure gauge. The second input transducer may be anything as long as it can acquire information indicating a salt concentration, for example, if the first input transducer 103 is a salinometer. The second input transducer may be anything as long as it can acquire information indicating a sugar concentration, for example, if the first input transducer 103 is a saccharimeter.

The second input transducer 202 generates a signal indicating the acquired environmental information. A signal to be generated by the second input transducer 202 may be any signal as long as the signal indicates the acquired environmental information and may be an electrical signal or an optical signal.

The output transducer 203 acts on the environment by executing a predetermined operation corresponding to the current value of the reinforcement learning data under control of the learning control unit 205 that is described later. The acting on the environment specifically means changing the environment. The output transducer 203 may be anything as long as it can execute the predetermined operation corresponding to the current value of the reinforcement learning data. The output transducer 203 may be a drive device, such as a motor, or an actuator for, e.g., an air conditioner or a printer. The output transducer 203 may be, for example, an output interface for a light-emitting device, such as a display or lighting, an odor generation device, a speaker, a force sense generation device, a vibration generation device, or the like.

The reward output unit 204 outputs a reward based on the environmental information acquired by the second input transducer 202 and the relationship information. The reward is a value (i.e., numerical emotional information) representing the magnitude of an emotion represented by emotional information associated, through the relationship information, with the environmental information acquired by the second input transducer 202.

The learning control unit 205 updates the reinforcement learning data stored in the second auxiliary storage device 201 based on the environmental information, the reward, and the current value of the reinforcement learning data. Specifically, the learning control unit 205 updates the reinforcement learning data such that an active operation corresponding to the reinforcement learning data after the updating does not result in reduction in reward.

The learning control unit 205 may update the reinforcement learning data by any method as long as the learning control unit 205 can update the reinforcement learning data based on the environmental information, the reward, and the current value of the reinforcement learning data such that an active operation corresponding to the reinforcement learning data after the updating does not result in reduction in reward. The learning control unit 205 may update the reinforcement learning data with, for example, a value determined by Q-learning using ε-greedy.

The updating of the reinforcement learning data by the learning control unit 205 means not lowering the accuracy of the control selection policy.

The learning control unit 205 controls operation of the output transducer 203 based on the control selection policy and the current value of the reinforcement learning data.

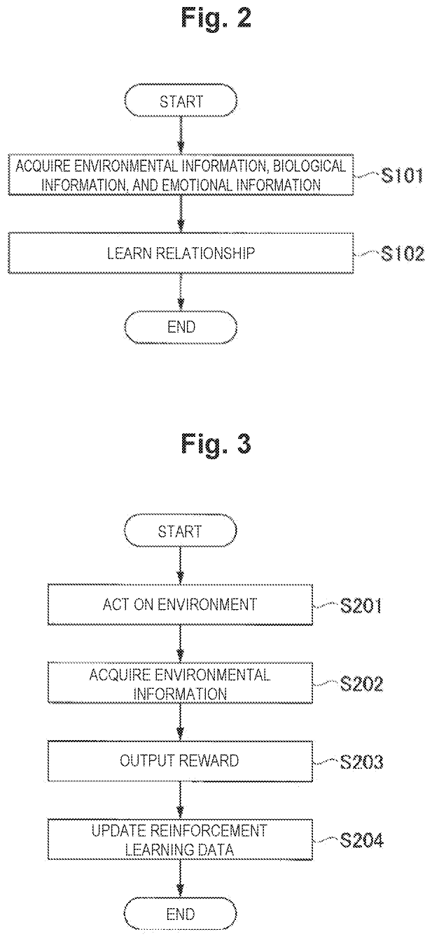

FIG. 2 is a flowchart showing the flow of a specific process by the first learning apparatus 10 according to the first embodiment.

The biological information acquisition unit 102 acquires biological information, the first input transducer 103 acquires environmental information, and the emotional information acquisition unit 104 acquires emotional information (step S101). The relationship information learning unit 105 learns, through machine learning, a relationship among the biological information, the environmental information, and the emotional information based on the acquired biological information, environmental information, and emotional information (step S102). The processes in steps S101 and S102 are repeated a predetermined number of times.

FIG. 3 is a flowchart showing the flow of a specific process by the second learning apparatus 20 according to the first embodiment.

The output transducer 203 acts on the environment under control of the learning control unit 205 that is based on the reinforcement learning data and the control selection policy stored in the second auxiliary storage device 201 (step S201). The second input transducer 202 acquires environmental information (step S202). The reward output unit 204 outputs a reward based on the environmental information acquired by the second input transducer 202 and relationship information (step S203). The learning control unit 205 updates the reinforcement learning data based on the environmental information, the reward, and the reinforcement learning data at the time of step S201 (step S204). After step S204, the processes in steps S201 to S204 are repeated a predetermined number of times.

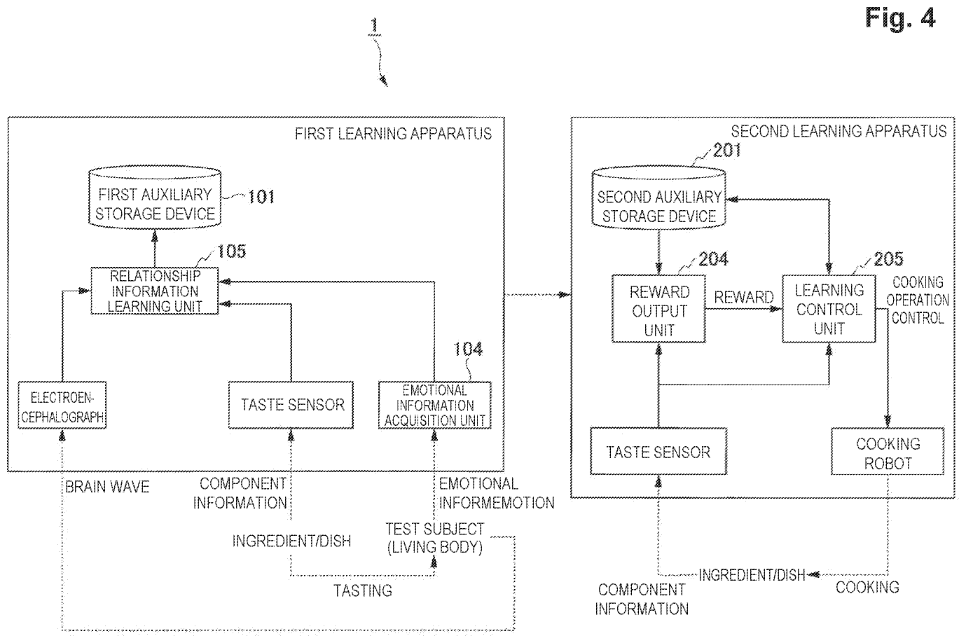

FIG. 4 is a diagram showing an example of application in which the learning system 1 according to the first embodiment is applied to learning of cooking by a cooking robot. Elements having the same functions as those in FIG. 1 are denoted by the same reference numerals in FIG. 4.

In the example of application in FIG. 4, an electroencephalograph is a specific example of the biological information acquisition unit 102. In the example of application in FIG. 4, a taste sensor in the first learning apparatus is a specific example of the first input transducer 103. In the example of application in FIG. 4, an ingredient/dish represents an ingredient or a dish and is a specific example of an environment. In the example of application in FIG. 4, component information is a specific example of environmental information. The component information is information related to components of a dish, such as a salt concentration and a sugar concentration. In the example of application in FIG. 4, tasting in the first learning apparatus is a specific example of an action. In the example of application in FIG. 4, the cooking robot is a specific example of the output transducer 203. In the example of application in FIG. 4, cooking operation control is a specific example of control. In the example of application in FIG. 4, cooking is a specific example of an action in the second learning apparatus. In the example of application in FIG. 4, a taste sensor in the second learning apparatus is a specific example of the second input transducer.

In the example of application in FIG. 4, the first learning apparatus acquires, with the electroencephalograph, brain waves that are biological information at the time of tasting by a taster (a test subject) of the ingredient/dish. In the example of application in FIG. 4, the first learning apparatus analyzes components of the ingredient/dish with the taste sensor and acquires an analysis result. In the example of application in FIG. 4, the first learning apparatus acquires, with the emotional information acquisition unit 104, emotional information indicating a like or dislike for the dish of the taster (test subject) of the ingredient/dish. The first learning apparatus learns, through machine learning, a relationship related to taste preferences of the taster (test subject) of the ingredient/dish based on the brain waves acquired by the electroencephalograph, a salt concentration acquired by the taste sensor, and the emotional information indicating the like or dislike acquired by the emotional information acquisition unit 104.

In the example of application in FIG. 4, the second learning apparatus learns, through machine learning, a reinforcement learning parameter that increases a reward based on the relationship learned by the first learning apparatus, the cooking by the cooking robot, and tasting by the taste sensor.

The learning system 1 according to the first embodiment with the above-described configuration includes the first learning apparatus 10 that determines relationship information (i.e., a reward function) including emotional information. Additionally, in the learning system 1 according to the first embodiment with the above-described configuration, the second learning apparatus 20 improves the accuracy of a control selection policy without intervention of a designer of the first learning apparatus 10 based on the relationship information.

It is thus possible to curb increase in labor of the designer associated with improvement in the accuracy of a control selection policy.

### Second Embodiment

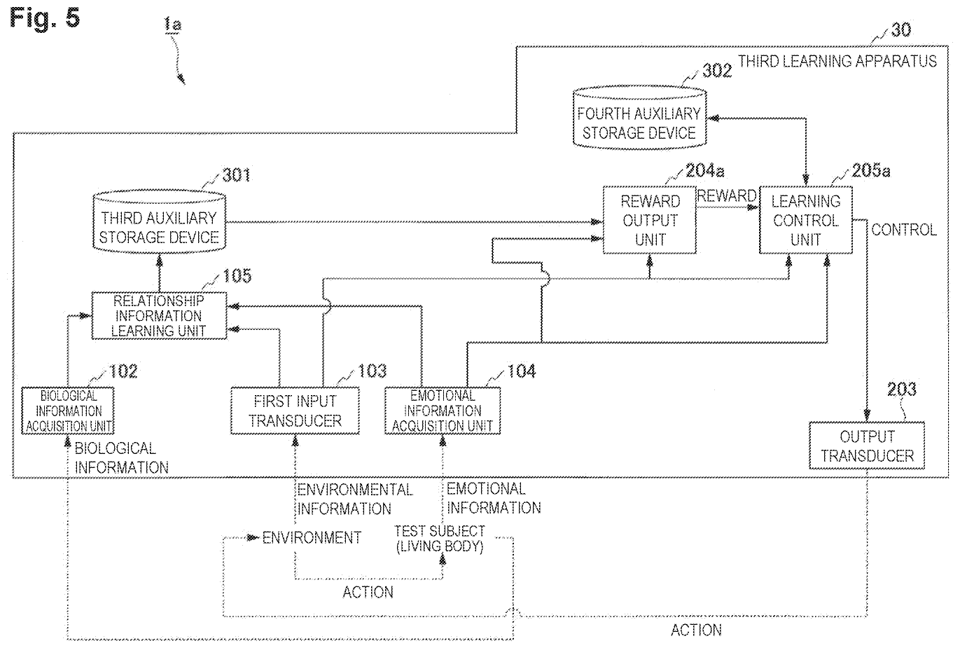

FIG. 5 is a diagram showing a specific example of a system configuration of a learning system 1a according to a second embodiment.

The learning system 1a includes a third learning apparatus 30. The third learning apparatus 30 includes a CPU (Central Processing Unit), a RAM (Random Access Memory), a third auxiliary storage device 301, a fourth auxiliary storage device 302, and the like that are connected by a bus and executes a program. The first learning apparatus 10 includes a biological information acquisition unit 102, a first input transducer 103, an emotional information acquisition unit 104, a relationship information learning unit 105, an output transducer 203, a reward output unit 204a, and a learning control unit 205a through the execution of the program.

Elements having the same functions as those in FIG. 1 are denoted by the same reference numerals, and a description thereof will be omitted below.

The third auxiliary storage device 301 is constructed using a storage device, such as a magnetic hard disk device or a semiconductor storage device. The third auxiliary storage device 301 stores relationship information. The relationship information is information indicating a relationship among biological information, environmental information, and emotional information.

The fourth auxiliary storage device 302 is constructed using a storage device, such as a magnetic hard disk device or a semiconductor storage device. The fourth auxiliary storage device 302 stores reinforcement learning data and a control selection policy.

The reward output unit 204a outputs a reward based on environmental information acquired by the first input transducer 103 and the relationship information. Note that the reward according to the second embodiment is a value (i.e., numerical emotional information) representing the magnitude of an emotion represented by emotional information associated, through the relationship information, with the environmental information acquired by the first input transducer 103.

The learning control unit 205a updates the reinforcement learning data stored in the fourth auxiliary storage device 302 based on the environmental information, the reward, and a current value of the reinforcement learning data. Specifically, the learning control unit 205a updates the reinforcement learning data such that an active operation corresponding to the reinforcement learning data after the updating does not result in reduction in reward.

The learning control unit 205a may update the reinforcement learning data by any method as long as the learning control unit 205a can update the reinforcement learning data based on the environmental information, the reward, and the current value of the reinforcement learning data such that an active operation corresponding to the reinforcement learning data after the updating does not result in reduction in reward. The learning control unit 205a may update the reinforcement learning data with, for example, a value determined by Q-learning using ε-greedy.

The updating of the reinforcement learning data by the learning control unit 205a means not lowering the accuracy of the control selection policy.

The learning control unit 205a also controls operation of the output transducer 203 based on the control selection policy and the current value of the reinforcement learning data.

Additionally, the learning control unit 205a outputs the reinforcement learning data after the updating to the relationship information learning unit 105.

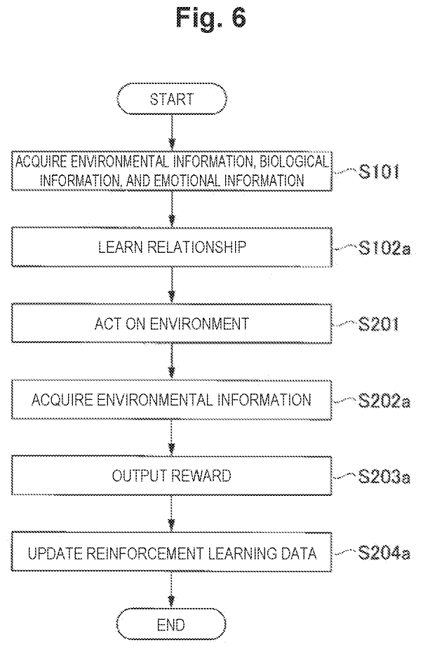

FIG. 6 is a flowchart showing the flow of a specific process by the third learning apparatus 30 according to the second embodiment.

The same processes as those in FIGS. 2 and 3 are denoted by the same reference numerals, and a description thereof will be omitted below.

Subsequently to step S101, the relationship information learning unit 105 learns, through machine learning, a relationship among biological information, environmental information, emotional information, and the reinforcement learning data based on the biological information, the environmental information, the emotional information, and the reinforcement learning data (step S102a). Subsequently to step S102a, step S201 is executed. Subsequently to step S201, the first input transducer 103 acquires environmental information (step S202a). The reward output unit 204a outputs a reward based on the relationship acquired in step S102a (step S203a). The learning control unit 205a updates the reinforcement learning data based on the environmental information, the reward, and the reinforcement learning data at the time of step S201 (step S204a).

After step S204, the processes in steps S101 to S204a in FIG. 6 are repeated a predetermined number of times.

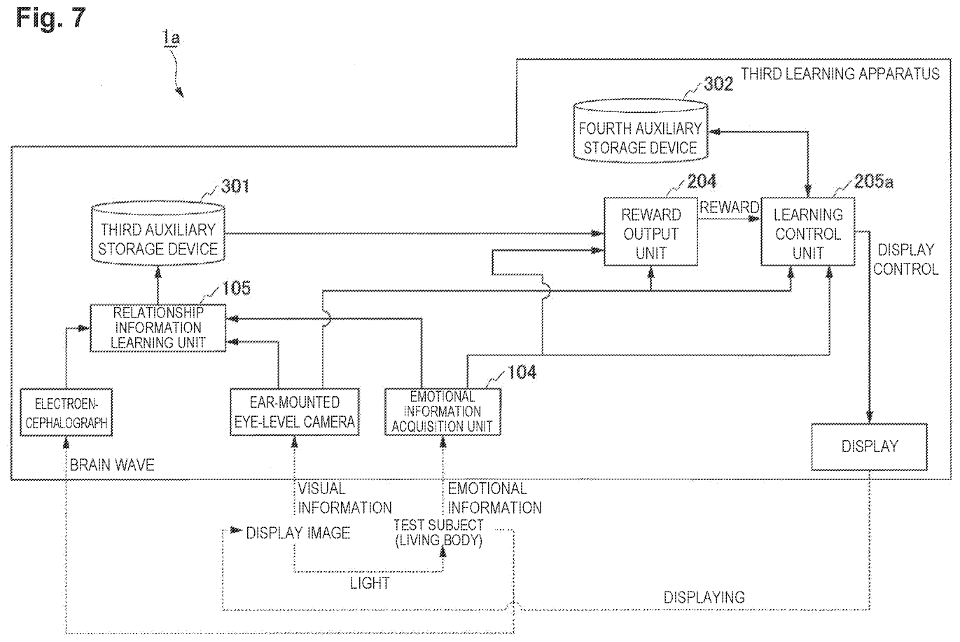

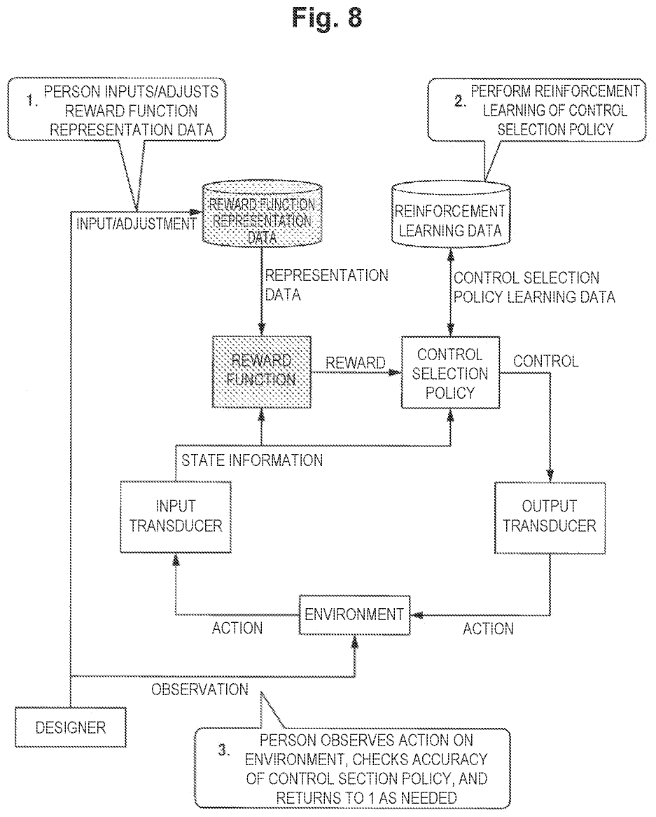

FIG. 7 is a diagram showing an example of application in which the learning system 1a according to the second embodiment is applied to learning of display screen control by an image display device. Elements having the same functions as those in FIG. 5 are denoted by the same reference numerals in FIG. 7.

In the example of application in FIG. 7, an electroencephalograph is a specific example of the biological information acquisition unit 102. In the example of application in FIG. 7, an ear-mounted eye-level camera in the third learning apparatus is a specific example of the first input transducer 103. The ear-mounted eye-level camera acquires visual information equivalent to that obtained at a test subject's eye level when used in a state of being mounted on ears of the test subject. In the example of application in FIG. 7, a display image is a specific example of an environment. In the example of application in FIG. 7, the visual information is a specific example of environmental information. In the example of application in FIG. 7, light is a specific example of an action of the environment on the test subject. The light represents incidence of light from a display screen on the user' eyes. In the example of application in FIG. 7, a display is a specific example of the output transducer 203. In the example of application in FIG. 7, display control is a specific example of control. In the example of application in FIG. 7, display is a specific example of an action of the output transducer 203 on the environment.

In the example of application in FIG. 7, the third learning apparatus acquires, with the electroencephalograph, brain waves that are biological information of a person (the test subject) at a position where the display image is viewable. In the example of application in FIG. 7, the third learning apparatus acquires, with the ear-mounted eye-level camera, the display image on a line of sight of the test subject as visual information. In the example of application in FIG. 7, the third learning apparatus acquires, with the emotional information acquisition unit 104, emotional information indicating a like or dislike of the person (test subject) at the position where the display image is viewable. The third learning apparatus performs reinforcement learning of control related to output image selection based on the brain waves acquired by the electroencephalograph, the visual information acquired by the ear-mounted eye-level camera, and the emotional information indicating the like or dislike acquired by the emotional information acquisition unit 104.

The learning system 1a according to the second embodiment with the above-described configuration includes the biological information acquisition unit 102, the first input transducer 103, the emotional information acquisition unit 104, the relationship information learning unit 105, the output transducer 203, the reward output unit 204, and the learning control unit 205a. It is thus possible to curb increase in labor of a designer associated with improvement in the accuracy of a control selection policy.

(Modification)

Note that the learning system 1 according to the first embodiment or the learning system 1a according to the second embodiment may be applied to a device that learns, through reinforcement learning, a massage method and a massage position in accordance with hardness of each body part and a brain-wave condition of a test subject. In this case, specifically, the output transducer 203 is a massaging chair, and the first input transducer 103 and the second input transducer 202 are each a force sensor.

Note that the learning system 1 and the learning system 1a may perform optimization, such as learning data classification using identification information of a test subject, a feature quantity of the test subject, a time, positioning information, and the like.

Note that the first learning apparatus 10 may be a device that is composed of one housing or a device that is composed of a plurality of divided housings. If the first learning apparatus 10 is composed of a plurality of divided housings, one (ones) of functions of the first learning apparatus 10 described above may be implemented at a position physically apart over a network.

Note that the second learning apparatus 20 may be a device that is composed of one housing or a device that is composed of a plurality of divided housings. If the second learning apparatus 20 is composed of a plurality of divided housings, one (ones) of functions of the second learning apparatus 20 described above may be implemented at a position physically apart over a network.

Note that the third learning apparatus 30 may be a device that is composed of one housing or a device that is composed of a plurality of divided housings. If the third learning apparatus 30 is composed of a plurality of divided housings, one (ones) of functions of the third learning apparatus 30 described above may be implemented at a position physically apart over a network.

Note that the first learning apparatus 10 and the second learning apparatus 20 need not be configured as separate devices and that the two may be in one housing.

Note that the third learning apparatus need not include the third auxiliary storage device 301 and the fourth auxiliary storage device 302 as different function units and may include the third auxiliary storage device 301 and the fourth auxiliary storage device 302 as one auxiliary storage device that stores relationship information, reinforcement learning data, and a control section policy.

Note that all or some of functions of the first learning apparatus 10, the second learning apparatus 20, and the third learning apparatus 30 may be implemented using hardware, such as an ASIC (Application Specific Integrated Circuit), a PLD (Programmable Logic Device), or an FPGA (Field Programmable Gate Array). A program may be recorded on a computer-readable recording medium. The computer-readable recording medium is, for example, a storage device, such as a portable medium (e.g., a flexible disk, a magnetoopical disk, a ROM, or a CD-ROM) or a hard disk incorporated in a computer system. The program may be transmitted via telecommunications lines.

Note that the relationship information learning unit 105 may further learn a relationship between biological information that has a predetermined degree or a higher degree of correlation with emotional information and the emotional information.

Note that the learning control units 205 and 205a are examples of a control unit. Note that the first learning apparatus 10, the second learning apparatus 20, and the third learning apparatus 30 are examples of a learning apparatus. Note that the first input transducer 103 is an example of a first environmental information acquisition unit. Note that the second input transducer 202 is an example of a second environmental information acquisition unit. Note that the output transducer 203 is an example of an output unit.

The embodiments of this invention have been described above in detail with reference to the drawings. A specific configuration is not limited to these embodiments, and a design and the like within a range not departing from the gist of this invention are also included.

### REFERENCE SIGNS LIST

1 Learning system
1a Learning system
10 First learning apparatus
20 Second learning apparatus
30 Third learning apparatus
101 First auxiliary storage device
102 Biological information acquisition unit
103 First input transducer
104 Emotional information acquisition unit
105 Relationship information learning unit
201 Second auxiliary storage device
202 Second input transducer
203 Output transducer
204 Reward output unit
205 Learning control unit
301 Third auxiliary storage device
302 Fourth auxiliary storage device
204a Reward output unit
205a Learning control unit

1. A learning apparatus comprising:
a processor; and
a storage medium having computer program instructions stored thereon,
when executed by the processor, perform to:
acquires biological information, the biological information being information indicating a vital reaction of a test subject to a predetermined environment;
an emotional information acquisition unit that acquires emotional information, the emotional information being information indicating an emotion of the test subject toward the environment;
a first environmental information acquisition unit that acquires environmental information, the environmental information being information indicating an attribute of the environment acting on the test subject; and
learns, through machine learning, a relationship among the biological information, the emotional information, and the environmental information based on the biological information, the emotional information, and the environmental information.

2. (canceled)

3. (canceled)

4. The learning apparatus according to claim 1, wherein the relationship information learning unit further learns a relationship between the biological information that has a predetermined degree or a higher degree of correlation with the emotional information and the emotional information.

5. A learning method comprising:
a biological information acquisition step of acquiring biological information that is information indicating a vital reaction of a test subject to a predetermined environment;
an emotional information acquisition step of acquiring emotional information that is information indicating an emotion of the test subject toward the environment;
a first environmental information acquisition step of acquiring environmental information that is information indicating an attribute of the environment acting on the test subject; and
a relationship information learning step of learning, through machine learning, a relationship among the biological information, the emotional information, and the environmental information based on the biological information, the emotional information, and the environmental information.

6. (canceled)

7. (canceled)

8. A non-transitory computer-readable medium having computer-executable instructions that, upon execution of the instructions by a processor of a computer, cause the computer to function as a learning apparatus according to claim 1.
